(19) United States
(12) Patent Application Publication
(10) Pub. No.: US 2003/0157150 A1
(43) Pub. Date: Aug. 21, 2003

(54) Formulation and process for manufacturing antimicrobial vinyl gloves

(76) Inventor: Che-Hao Lee, Shanghai (CN)

Correspondence Address: MADSON & METCALF, GATEWAY TOWER WEST, SUITE 900, 15 WEST SOUTH TEMPLE, SALT LAKE CITY, UT, 84101

(21) Appl. No.: 10/364,852

(22) Filed: Feb. 11, 2003

Related U.S. Application Data
(60) Provisional application No. 60/356,281, filed on Feb. 19, 2002.

(51) Int. Cl. A61K 9/70; C08K 3/00

(52) U.S. Cl. 424/443; 523/122

(57) ABSTRACT

A formulation for manufacturing an antimicrobial article is provided. The formulation comprises a PVC resin, a plasticizer blend, a stabilizer, a powdered antimicrobial agent, a surfactant and a dispersing agent. A process for manufacturing an antimicrobial PVC glove is also provided. Firstly, an effective amount of a powered antimicrobial agent is allowed to suspend in a polymer plastisol including a PVC resin, a plasticizer blend, a stabilizer, a surfactant and a dispersing agent. Then, a shape of the glove is dipped into a mixture of the polymer plastisol and powered antimicrobial agent. Then, the mixture on the shape is cured so as to form the glove.

### RELATED APPLICATION

This application claims the benefit of U.S. Provisional Application No. 60/356,281 filed Feb. 19, 2002 and entitled Formulation and Process for Manufacturing Antimicrobial Vinyl Gloves, which is incorporated herein by reference.

### FIELD OF THE INVENTION

The present invention relates to a formulation and a process for manufacturing a glove, and more particularly to a formulation and a process for manufacturing an antimicrobial glove.

### BACKGROUND OF THE INVENTION

Protective gloves are widely used in hospitals, pharmaceutical plants, food plants, kitchens or even public places. Gloves are generally made of a polymer resin. For example, a so-called vinyl glove is produced by using polyvinyl chloride (PVC) as a main component. Conventionally, the use of a protective glove isolates bacteria from user's hand so as to reduce the risk of bacterial contamination. Since the bacteria attached to the surface of the glove are not killed, bacteria or other microbes may grow on the glove surface. Therefore, the glove might become a newly contaminating source.

Especially when the gloves are used in the food industries, it is desirable to provide continuous bacterial protection during glove's use. For a purpose of achieving continuous bacterial protection, antimicrobial gloves are developed by dipping glove shapes in a PVC plastisol containing a PVC resin, an antimicrobial agent, a plasticizer and a stabilizer. Please refer to U.S. Pat. Nos. 5,888,441 and 5,906,823, which are incorporated herein for reference, to realize examples of polymer resins, additives and method for producing a glove. Generally, in order to obtain suitable physical properties of these gloves, for example thickness, strength, elasticity, deformation, etc., the PVC plastisol needs to be elaborately controlled before the dipping step is effected. In addition, according to the manufacturing process of the prior art, the amount of the antimicrobial agent is limited to a low level (e.g. less than 1 weight percent) in order not to impair the physical properties of the gloves. As a result, the antimicrobial agent will diminish soon since the glove is used because it will gradually disappear from the glove surfaces and cannot be replenished.

### SUMMARY OF THE INVENTION

It is an object of the present invention to provide an antimicrobial glove having a sustained antimicrobial effect and suitable physical properties.

A first aspect of the present invention relates to a formulation for manufacturing an antimicrobial article. The formulation comprises 80 to 120 weight parts of a PVC resin, 35 to 125 weight parts of a plasticizer blend, 1 to 5 weight parts of a stabilizer, 1 to 12% by weight of a powdered antimicrobial agent relative to the total weight of the PVC resin, the plasticizer blend and the stabilizer, 0.1 to 1% by weight of a surfactant relative to the total weight of the PVC resin, the plasticizer blend and the stabilizer, and 0.01 to 0.12% by weight of a dispersing agent relative to the total weight of the PVC resin, the plasticizer blend and the stabilizer.

In an embodiment, the plasticizer blend comprises 10 to 30 weight part of 2,2,4-trimethyl-1,3-pentanediol diisobutyrate (TXIB) and 25 to 95 weight part of a plasticizer selected from a group consisting of dioctyl phthalate (DOP), diisononyl phthalate (DINP), dioctyl terephthalate (DOTP), butyl benzyl phthalate (BBP) and a combination thereof.

In an embodiment, the plasticizer blend comprises 15 to 25 weight part of 2,2,4-trimethyl-1,3-pentanediol diisobutyrate (TXIB) and 80 to 90 weight part of a plasticizer selected from a group consisting of dioctyl phthalate (DOP), diisononyl phthalate (DINP), dioctyl terephthalate (DOTP), butyl benzyl phthalate (BBP) and a combination thereof.

In an embodiment, the stabilizer is a Ca—Zn stabilizer.

In an embodiment, the powdered antimicrobial agent is activated by light and offers a sustained release of chlorine dioxide from the article.

In an embodiment, the article is a glove.

A second aspect of the present invention relates to a process for manufacturing an antimicrobial article. The process comprises step of allowing an effective amount of a powered antimicrobial agent to suspend in a polymer plastisol, dipping a shape of the article into the resulting mixture of the polymer plastisol and the powered antimicrobial agent, and curing the mixture on the shape so as to form the article.

In an embodiment, the process further comprises a step of continuously stirring the mixture of the polymer plastisol and the powered antimicrobial agent before and upon the dipping step is performed.

In an embodiment, the powdered antimicrobial agent is activated by light and offers a sustained release of chlorine dioxide from the article.

In an embodiment, all steps of manufacturing the article are performed under an environment having an illuminance below 30 lux.

In an embodiment, the article is used under an environment having an illuminance above 30 lux so as to offer the article an antimicrobial effect.

In an embodiment, the polymer plastisol comprises 80 to 120 weight parts of a PVC resin, 35 to 125 weight parts of a plasticizer blend, 1 to 5 weight parts of a stabilizer, 0.1 to 1% by weight of a surfactant relative to the total weight of the PVC resin, the plasticizer blend and the stabilizer, and 0.01 to 0.12% by weight of a dispersing agent relative to the total weight of the PVC resin, the plasticizer blend and the stabilizer.

In an embodiment, the amount of the powdered antimicrobial agent is 1 to 12% by weight relative to the total weight of the PVC resin, the plasticizer blend and the stabilizer.

In an embodiment, the plasticizer blend comprises 10 to 30 weight part of 2,2,4-trimethyl-1,3-pentanediol diisobutyrate (TXIB) and 25 to 95 weight part of a plasticizer selected from a group consisting of dioctyl phthalate (DOP), diisononyl phthalate (DINP), dioctyl terephthalate (DOTP), butyl benzyl phthalate (BBP) and a combination thereof.

In an embodiment, the plasticizer blend comprises 15 to 25 weight part of 2,2,4-trimethyl-1,3-pentanediol diisobutyrate (TXIB) and 80 to 90 weight part of a plasticizer selected from a group consisting of dioctyl phthalate (DOP), diisononyl phthalate (DINP), dioctyl terephthalate (DOTP), butyl benzyl phthalate (BBP) and a combination thereof.

A third aspect of the present invention relates to a process for manufacturing an antimicrobial PVC glove. Firstly, an effective amount of a powered antimicrobial agent is allowed to suspend in a polymer plastisol including a PVC resin, a plasticizer blend, a stabilizer, a surfactant and a dispersing agent. Then, a shape of the glove is dipped into a mixture of the polymer plastisol and powered antimicrobial agent. Then, the mixture on the shape is cured so as to form the glove.

The above objects and advantages of the present invention will become more readily apparent to those ordinarily skilled in the art after reviewing the following detailed description.

### DETAILED DESCRIPTION OF THE PREFERRED EMBODIMENT

The term “antimicrobial” used herein refers to the inhibition of the growth of bacteria or other microbes on a glove surface, or the killing of bacteria or other microbes on a glove surface. Such bacteria or other microbes include but not limited to anthrax, E.coli, salmonella, listeria, Pseudomonas aeruginosa and Staphyloccus aureus.

The formulation suitable for manufacturing an antimicrobial glove of the present invention comprises the following components: (a) 80 to 120 weight parts of a PVC resin; (b) 35 to 125 weight parts of a plasticizer blend; (c) 1 to 5 weight parts of a stabilizer; (d) 1 to 12% by weight, and preferably 3 to 8% by weight of a powdered antimicrobial agent relative to the total weight of said PVC resin, said plasticizer blend and said stabilizer; (e) 0.1 to 1% by weight, and preferably 0.1 to 0.5% by weight of a surfactant relative to the total weight of said PVC resin, said plasticizer blend and said stabilizer; and (f) 0.01 to 0.12% by weight, and preferably 0.01 to 0.05 by weight of a dispersing agent relative to the total weight of said PVC resin, said plasticizer blend and said stabilizer.

The plasticizer blend (b) comprises 10 to 30 weight part of 2,2,4-trimethyl-1,3-pentanediol diisobutyrate (TXIB) and 25 to 95 weight part of a plasticizer selected from a group consisting of dioctyl phthalate (DOP), diisononyl phthalate (DINP), dioctyl terephthalate (DOTP), butyl benzyl phthalate (BBP) and a combination thereof. In a preferred embodiment, plasticizer blend (b) comprises 15 to 25 weight part of 2,2,4-trimethyl-1,3-pentanediol diisobutyrate (TXIB) and 80 to 90 weight part of a plasticizer selected from a group consisting of dioctyl phthalate (DOP), diisononyl phthalate (DINP), dioctyl terephthalate (DOTP), butyl benzyl phthalate (BBP) and a combination thereof. Such plasticizers are employed to enhance plasticity and flowability of the formulation during processing and molding, and offers softness and toughness to the final product.

The stabilizer (c) may be any suitable product that has been approved for use in the food industry, such as Ca—Zn stabilizer.

The powdered antimicrobial agent (d) used in the present invention is activated by light and offers a sustained release of chlorine dioxide from the glove. It is known that chlorine dioxide is a strong antimicrobial agent to kill most bacteria. Such powdered antimicrobial agent (d) can be commercially available from Bernard Technologies Inc. under the trademark of microlite®. Since such antimicrobial agent is activated by light, all steps of manufacturing the glove should be performed under low illuminance, for example below 30 lux. In other words, the glove is used under an environment having an illuminance above 30 lux so as to offer an antimicrobial effect. Since the amount of the powdered antimicrobial agent in present from 1 to 12% by weight of the glove, the antimicrobial effect is extremely long.

The surfactant (e) and the dispersing agent (f) used in the present invention are commercially available from Loveland Industries, Inc., U.S.A. under the trademark of EZ-MX 1 and EZ-MX 2, respectively. The surfactant (e) facilitates reducing surface tension of the plastisol and increasing emulsifying, wetting and dispersing properties of the powdered antimicrobial agent. The dispersing agent (f) is advantageous for enhancing dispersing effects of the powdered antimicrobial agent.

Conventionally, a glove is formed by using a conventional dipping process, i.e. by dipping a glove shape into a plastisol, removing the shape, and curing the plastisol, as described in U.S. Pat. No. 5,888,441. The process for manufacturing the vinyl glove according to the present invention is distinguished from that of prior art. Firstly, a polymer plastisol including the PVC resin (a), the plasticizer blend (b), the stabilizer (c), the surfactant (e) and the dispersing agent (f) is prepared. The polymer plastisol is transferred to a specified stirring tank. With continuously stirring, the powdered antimicrobial agent (d) is mixed into the polymer plastisol to be encapsulated by the polymer plastisol, thereby suspending in the polymer plastisol. In order to control viscosity and flowing property of the mixture to avoid powder sedimentation, the mixture should be continuously stirred. A glove shape is then dipped into the mixture of the polymer plastisol and the suspended antimicrobial agent. The thickness of the coating depends upon the viscosity of the mixture and the contact time of the shape and the mixture. Then, the shape is removed from the mixture and cured so as to form a glove. Additional post-treatments to be done, such as drying, cooling, cuff beading and stripping, are well-known in the art and need not to be described herein. The thickness of the glove is ranged from 0.04 to 0.25 mm. In order to keep all steps of manufacturing the glove under low illuminance, some suitable measures should be taken. For example, the workplaces for manufacturing the glove are sheltered by black cloth, and red bulbs are employed to replace ordinary ones. Moreover, the finished glove should also be packaged and stored under low illuminance, for example below 30 lux.

The gloves manufactured from the present invention could provide effective and continuous antimicrobial protection. In addition, the physical properties of the glove are not significantly reduced.

While the invention has been described in terms of what is presently considered to be the most practical and preferred embodiments, it is to be understood that the invention needs not be limited to the disclosed embodiment. On the contrary, it is intended to cover various modifications and similar arrangements included within the spirit and scope of the appended claims which are to be accorded with the broadest interpretation so as to encompass all such modifications and similar structures.

What is claimed is:

1. A formulation for manufacturing an antimicrobial article, said formulation comprising:
80 to 120 weight parts of a PVC resin;
35 to 125 weight parts of a plasticizer blend;
1 to 5 weight parts of a stabilizer;
1 to 12% by weight of a powdered antimicrobial agent relative to the total weight of said PVC resin, said plasticizer blend and said stabilizer;
0.1 to 1% by weight of a surfactant relative to the total weight of said PVC resin, said plasticizer blend and said stabilizer; and
0.01 to 0.12% by weight of a dispersing agent relative to the total weight of said PVC resin, said plasticizer blend and said stabilizer.

2. The formulation according to claim 1 wherein said plasticizer blend comprises 10 to 30 weight part of 2,2,4-trimethyl-1,3-pentanediol diisobutyrate (TXIB) and 25 to 95 weight part of a plasticizer selected from a group consisting of dioctyl phthalate (DOP), diisononyl phthalate (DINP), dioctyl terephthalate (DOTP), butyl benzyl phthalate (BBP) and a combination thereof.

3. The formulation according to claim 1 wherein said plasticizer blend comprises 15 to 25 weight part of 2,2,4-trimethyl-1,3-pentanediol diisobutyrate (TXIB) and 80 to 90 weight part of a plasticizer selected from a group consisting of dioctyl phthalate (DOP), diisononyl phthalate (DINP), dioctyl terephthalate (DOTP), butyl benzyl phthalate (BBP) and a combination thereof.

4. The formulation according to claim 1 wherein said stabilizer is a Ca—Zn stabilizer.

5. The formulation according to claim 1 wherein said powdered antimicrobial agent is activated by light and offers a sustained release of chlorine dioxide from said article.

6. The formulation according to claim 1 wherein said article is a glove.

7. A process for manufacturing an antimicrobial article comprising steps of:
allowing an effective amount of a powered antimicrobial agent to suspend in a polymer plastisol;
dipping a shape of said article into a mixture of said polymer plastisol and said powered antimicrobial agent; and
curing said mixture on said shape so as to form said article.

8. The process according to claim 7 further comprising a step of continuously stirring said mixture of said polymer plastisol and said powered antimicrobial agent before and upon said dipping step is performed.

9. The process according to claim 7 wherein said powdered antimicrobial agent is activated by light and offers a sustained release of chlorine dioxide from said article.

10. The process according to claim 9 wherein all steps of manufacturing said article are performed under an environment having an illuminance below 30 lux.

11. The process according to claim 9 wherein said article is used under an environment having an illuminance above 30 lux so as to offer said article an antimicrobial effect.

12. The process according to claim 7 wherein said polymer plastisol comprises:
80 to 120 weight parts of a PVC resin;
35 to 125 weight parts of a plasticizer blend;
1 to 5 weight parts of a stabilizer;
0.1 to 1% by weight of a surfactant relative to the total weight of said PVC resin, said plasticizer blend and said stabilizer; and
0.01 to 0.12% by weight of a dispersing agent relative to the total weight of said PVC resin, said plasticizer blend and said stabilizer.

13. The process according to claim 12 wherein the amount of said powdered antimicrobial agent is 1 to 12% by weight relative to the total weight of said PVC resin, said plasticizer blend and said stabilizer.

14. The process according to claim 12 wherein said plasticizer blend comprises 10 to 30 weight part of 2,2,4-trimethyl-1,3-pentanediol diisobutyrate (TXIB) and 25 to 95 weight part of a plasticizer selected from a group consisting of dioctyl phthalate (DOP), diisononyl phthalate (DINP), dioctyl terephthalate (DOTP), butyl benzyl phthalate (BBP) and a combination thereof.

15. The process according to claim 12 wherein said plasticizer blend comprises 15 to 25 weight part of 2,2,4-trimethyl-1,3-pentanediol diisobutyrate (TXIB) and 80 to 90 weight part of a plasticizer selected from a group consisting of dioctyl phthalate (DOP), diisononyl phthalate (DINP), dioctyl terephthalate (DOTP), butyl benzyl phthalate (BBP) and a combination thereof.

16. The process according to claim 12 wherein said stabilizer is a Ca—Zn stabilizer.

17. The process according to claim 7 wherein said article is a glove.

18. A process for manufacturing an antimicrobial PVC glove comprising steps of:
allowing an effective amount of a powered antimicrobial agent to suspend in a polymer plastisol including a PVC resin, a plasticizer blend, a stabilizer, a surfactant and a dispersing agent;
dipping a shape of said glove into a mixture of said polymer plastisol and said powered antimicrobial agent; and
curing said mixture on said shape so as to form said glove.

19. The process according to claim 18 wherein all steps of manufacturing said glove are performed under an environment having an illuminance below 30 lux.

20. The process according to claim 18 wherein said glove is used under an environment having an illuminance above 30 lux so as to offer said glove an antimicrobial effect.
